(19) United States
(12) Patent Application Publication
(10) Pub. No.: US 2024/0269109 A1
(43) Pub. Date: Aug. 15, 2024

(54) LIDOCAINE OR ARTICAINE FOR TREATING COVID-19, AUTOIMMUNE DISEASE OR CYTOKINE STORM RESPONSE

(71) Applicant: STICHTING MEDISCHE KLINIEK VELSEN, Velsen-Noord (NL)

(72) Inventors: Cornelis Jan Oostwouder, Velsen-Noord (NL); Robert Chanderprekash Jaikrishne, Velsen-Noord (NL)

(21) Appl. No.: 18/566,968

(22) PCT Filed: Jun. 1, 2022
(86) PCT No.: PCT/IB2022/055134

§ 371 Date: Dec. 8, 2022

(30) Foreign Application Priority Data
Jun. 3, 2021 (GB) 2107966.0

(51) Int. Cl. A61K 31/381; A61K 9/00; A61K 31/167; A61K 47/10; A61P 11/00; A61P 37/06

(52) U.S. Cl. CPC A61K 31/381; A61K 9/006; A61K 31/167; A61K 47/10; A61P 11/00; A61P 37/06

(57) ABSTRACT

A composition including lidocaine or a salt thereof, or articaine or a salt thereof, is disclosed for use in the treatment of COVID-19 or an autoimmune disease or a condition in which an immune response caused by a disease or infection causes a cytokine storm. A pharmaceutical formulation including lidocaine, or a salt thereof, or articaine, or a salt thereof is disclosed. The pharmaceutical formulation is formulated to facilitate uptake by a lymphatic system with one or more suitable excipients.

### CROSS-REFERENCE TO RELATED APPLICATIONS

This application claims priority to International Patent Application No. PCT/IB2022/055134 filed Jun. 1, 2022, which also claims priority to Great Britian Patent Application GB 2107966.0 filed Jun. 3, 2021, the contents of each of which is hereby incorporated by reference in its entirety.

### TECHNICAL FIELD

The present invention relates to a composition and method for treating an autoimmune disease or a condition in which an immune response caused by a disease or infection needs treating. Such an immune response is one causing hyperinflammation, such as, but not limited to a cytokine storm, giving rise to Acute Respiration Distress Syndrome (ARDS) or Cytokine Storm Syndrome (CSS) as can occur in SARS-COV-2 disease (COVID-19). It further relates to the treatment of systemic inflammation (including hyperinflammation) and the symptoms thereof post infection, such as occurs in Long-covid.

### BACKGROUND

The COVID-19 pandemic has meant many groups have been urgently looking at the repurposing of approved drugs that might address the symptoms of COVID-19 and the longer term effects resulting from infection.

Acute respiratory distress syndrome (ARDS) is the main cause of morbidity and mortality in Coronavirus disease 19 (Covid-19) for which, as of now, there is no effective treatment besides respiratory support and the use of high dose dexamethasone.

Many drugs, for example chloroquine and ivermectin, have been studied on hospitalized patients with severe manifestations of SARS-COV-2 infection, but none have shown to be successful.

ARDS is caused, and sustained, by an uncontrolled inflammatory activation characterized by a massive release of cytokines (cytokine storm), diffuse lung edema, inflammatory cell infiltration and disseminated coagulation. Macrophage and T lymphocyte dysfunction plays a central role in this syndrome.

In several experimental in vitro and in vivo models, many of these pathophysiological changes are triggered by stimulation of the P2X7 receptor.

In this regards, viral pathogens in the respiratory tract cause cellular stress and a subsequent innate immune response. This causes massive exocytosis of ATP, resulting in high extracellular ATP concentrations. Initially, this stimulates the purinergic P2Y2 and P2X4 receptors, resulting in a short period of surfactant exocytosis, but as ATP levels continue to rise, the P2X4 and P2Y2 receptors become insensitive, and the normal release of surfactant is prevented. When the extracellular levels of ATP start to exceed the lower threshold for the activation of the P2X7 extracellular ATP receptors (P2X7Rs) located on the cell surface of the innate immune cells, it causes a pro-inflammatory response of the innate immunity, followed by a massive release of inflammatory mediators and a subsequent cytokine storm. The resulting vascular leakage and pulmonary edema induce the disaggregation and inactivation of pulmonary surfactant, a key element in the pathogenesis of ARDS that ends in alveolar collapse and reduced gas exchange.

Applicant hypothesized that this P2X7 receptor might be an ideal candidate to target in Covid-19-associated ARDS and Long-covid. Unfortunately, most of the known P2X7 receptor blockers become toxic when given to a patient at dose levels required for effective treatment.

They hypothesised that auto immune diseases might also be treated by the same approach—namely, blocking of the P2X7 receptor, using lidocaine or very similar drugs, such as, articaine at an effective dose. Articaine contains a thioprene ring rather than a benzene ring, which has the benefit of enhanced lipid solubility and thus increased diffusion making it more potent (×1.5) and less toxic (0.6).

Prior art identified includes the following:
Trials, vol 22, no 1, 2021 Muller Marie et al “impact of intravenous lidocaine on clinical outcomes of patients with ARDS during COVID-19 pandemia (LidoCovid): A structured summary of a study protocol for a randomised control trial” p. 131;
Bone Marrow Transplantation, vol 28, no 1, Voltarelli J C et al., “Beneficial effect of intravenous lidocaine in cutaneous chronic graft-versus-host disease secondary to donor lymphocyte infusion”, p. 97-99;
LUNG, vol 182, no 1, 2004 Huang T K et al, “Surfactant lavage with Lidocaine improves pulmonary function in piglets after HCl-induced acute lung injury”, p15-25;
RU 2742505;
CN 111150738;
CN 103142643; and
CN 104127397.

A first object of the invention was to identify a drug or drugs for use in treating an autoimmune disease or a condition in which an immune response caused by a disease or infection needs treating.

A second and further object was to formulate such a drug for such use.

A third and further object was to deliver such drugs in a manner, and at a dose, which facilitated effective treatment.

### SUMMARY

In accordance with a first aspect of the present invention there is provided lidocaine or a salt thereof or articaine or a salt thereof for use in the treatment of an autoimmune disease or a condition in which an immune response caused by a disease or infection causes hyperinflammation.

In one embodiment it causes a cytokine storm.

The autoimmune disease may be any of those listed below:
Achalasia
Addison's disease
Adult Still's disease
Agammaglobulinemia
Alopecia areata
Amyloidosis
Ankylosing spondylitis
Anti-GBM/Anti-TBM nephritis
Antiphospholipid syndrome
Autoimmune angioedema
Autoimmune dysautonomia
Autoimmune encephalomyelitis
Autoimmune hepatitis
Autoimmune inner ear disease (AIED)
Autoimmune myocarditis
Autoimmune oophoritis
Autoimmune orchitis
Autoimmune pancreatitis
Autoimmune retinopathy
Autoimmune urticaria
Axonal & neuronal neuropathy (AMAN)
Baló disease
Behcet's disease
Benign mucosal pemphigoid
Bullous pemphigoid
Castleman disease (CD)
Celiac disease
Chagas disease
Chronic inflammatory demyelinating polyneuropathy (CIDP)
Chronic recurrent multifocal osteomyelitis (CRMO)
Churg-Strauss Syndrome (CSS) or Eosinophilic Granulomatosis (EGPA)
Cicatricial pemphigoid
Cogan's syndrome
Cold agglutinin disease
Congenital heart block
Coxsackie myocarditis
CREST syndrome
Crohn's disease
Dermatitis herpetiformis
Dermatomyositis
Devic's disease (neuromyelitis optica)
Discoid lupus
Dressler's syndrome
Endometriosis
Eosinophilic esophagitis (EoE)
Eosinophilic fasciitis
Erythema nodosum
Essential mixed cryoglobulinemia
Evans syndrome
Fibromyalgia
Fibrosing alveolitis
Giant cell arteritis (temporal arteritis)
Giant cell myocarditis
Glomerulonephritis
Goodpasture's syndrome
Granulomatosis with Polyangiitis
Graves' disease
Guillain-Barre syndrome
Hashimoto's thyroiditis
Hemolytic anemia
Henoch-Schonlein purpura (HSP)
Herpes gestationis or pemphigoid gestationis (PG)
Hidradenitis Suppurativa (HS) (Acne Inversa)
Hypogammaglobulinemia
IgA Nephropathy
IgG4-related sclerosing disease
Immune thrombocytopenia purpura (ITP)
Inclusion body myositis (IBM)
Interstitial cystitis (IC)
Juvenile arthritis
Juvenile diabetes (Type 1 diabetes)
Juvenile myositis (JM)
Kawasaki disease
Lambert-Eaton syndrome
Leukocytoclastic vasculitis
Lichen planus
Lichen sclerosus
Ligneous conjunctivitis
Linear IgA disease (LAD)
Lupus
Lyme disease chronic
Meniere's disease
Microscopic polyangiitis (MPA)
Mixed connective tissue disease (MCTD)
Mooren's ulcer
Mucha-Habermann disease
Multifocal Motor Neuropathy (MMN) or MMNCB
Multiple sclerosis
Myasthenia gravis
Myelin Oligodendrocyte Glycoprotein Antibody Disorder
Myositis
Narcolepsy
Neonatal Lupus
Neuromyelitis optica
Neutropenia
Ocular cicatricial pemphigoid
Optic neuritis
Palindromic rheumatism (PR)
PANDAS
Paraneoplastic cerebellar degeneration (PCD)
Paroxysmal nocturnal hemoglobinuria (PNH)
Parry Romberg syndrome
Pars planitis (peripheral uveitis)
Parsonage-Turner syndrome
Pemphigus
Peripheral neuropathy
Perivenous encephalomyelitis
Pernicious anemia (PA)
POEMS syndrome
Polyarteritis nodosa
Polyglandular syndromes type I, II, III
Polymyalgia rheumatica
Polymyositis
Postmyocardial infarction syndrome
Postpericardiotomy syndrome
Primary Biliary Cholangitis
Primary sclerosing cholangitis
Progesterone dermatitis
Psoriasis
Psoriatic arthritis
Pure red cell aplasia (PRCA)
Pyoderma gangrenosum
Raynaud's phenomenon
Reactive Arthritis
Reflex sympathetic dystrophy
Relapsing polychondritis
Restless legs syndrome (RLS)
Retroperitoneal fibrosis
Rheumatic fever
Rheumatoid arthritis
Sarcoidosis
Schmidt syndrome
Scleritis
Scleroderma
Sjögren's syndrome
Sperm & testicular autoimmunity
Stiff person syndrome (SPS)
Subacute bacterial endocarditis (SBE)
Susac's syndrome
Sympathetic ophthalmia (SO)
Takayasu's arteritis
Temporal arteritis/Giant cell arteritis
Thrombocytopenia purpura (TTP)
Thyroid eye disease (TED)
Tolosa-Hunt syndrome (THS)
Transverse myelitis
Type 1 diabetes
Ulcerative colitis (UC)
Undifferentiated connective tissue disease (UCTD)
Uveitis
Vasculitis
Vitiligo
Vogt-Koyanagi-Harada Disease

Of these inflammatory diseases, those in which Cytokine Storm Syndrome presents, are particular targets for treatment.

Particularly favoured diseases for treatment are: ARDS, covid-19 and Long-covid.

Lidocaine, also known as lignocaine is sold under the brand name Xylocaine®. It is a local anaesthetic of the amino amide type.

As an approved medicine it is used intravenously, subcutaneously, topically and orally.

However, in these forms it is not particularly suited to targeting the lymphatic system and treating hyperinflammation by blocking the P2X7 receptors, thus stopping the “cytokine storm” which can be the cause of life-threatening ARDS in SARS-COV-2 disease (COVID-19) and other autoimmune diseases.

A related drug, Articaine is available in Europe with or without adrenaline. The epinephrine-free (adrenaline-free) version is sold under the brand name Ultracain D (4%). The version with epinephrine (adrenaline) is available in Europe under the brand name Supracain 4% with an epinephrine concentration of 1:200,000

Thus, in accordance with a second aspect of the present invention there is provided a pharmaceutical formulation comprising lidocaine or a salt thereof or articaine or a salt thereof which is formulated to facilitate uptake by the lymphatic system by the incorporation of one or more suitable excipients.

Furthermore, it is preferably packaged in a delivery form, and with instructions, for oromucosal or sublingual administration.

The delivery form is preferably a unit dose in a volume of less than or equal to 1 ml, and more preferably still less than or equal to 0.5 ml which contains a high concentration of lidocaine or a salt thereof or articaine or a salt thereof as compared to currently approved dosage forms of these drugs.

Thus, a unit dose of lidocaine contains from 60-100 mg and a unit dose of articaine contains from 40-100 mg.

Preferably, though not essentially, the Lidocaine or Articaine, or a salt thereof is dissolved in one or more lipophilic excipients although it is possible to achieve uptake using polar solvents.

The use of long chain fatty acids and/or surface modifiers such as polyethylene glycol (PEG) and/or surfactants are preferred.

Favoured delivery systems include, but are not limited to, nanoparticles, microspheres, liposomes, emulsifying drug delivery systems (EDDS) and variants thereof e.g. self-emulsifying drug delivery systems (SEDDS), self-micro emulsifying drug delivery systems (SMEDDS) and self-nano emulsifying drug delivery systems (SNEDDS).

The simplest forms are drugs in oils such as medium chain triglycerides.

Thus, suitable formulations may include functional excipients, such as lipophilic and polar solvents, co-solvents, viscosity modifiers, surfactants, sweeteners and flavourants.

To make it palatable the formulation includes a sweetener, e.g. saccharin and a flavourant, e.g. a banana flavour.

To facilitate their solubility in a low volume unit dose, (less than or equal to 1 ml and more preferably still less than or equal to 0.5 ml) a polar solvent is also used. A preferred polar solvent is ethanol.

Additionally, one or more co-solvents are used. Suitable co-solvents include polyethylene glycol (Macrogol 400) and propylene glycol (E 1520).

A problem with using, particularly, Lidocaine is its' known toxicity. Indeed, a benefit of Articaine is it is less toxic and more potent than Lidocaine.

Local anesthetics, such as lidocaine, can cause methemoglobinemia. This can occur when administering the lidocaine at high doses, typically more than 4-5 mg/kg bodyweight for local anesthesia or plasma levels>5 mg/L.

By administering the lidocaine or articaine oromucosally or sublingually, absorption mainly take place by the lymphatic system, and not the venous system. This avoids toxic plasma levels and hepatic first pass elimination, resulting in low plasma levels of lidocaine or articaine, thereby avoiding methemoglobinemia.

Finding an effective dose without causing toxicity for either drug was essential.

To achieve an IC50 for blocking the P2X7 receptor, a plasma concentration of 0.3 mM (66.07 μg/ml) is required. By contrast, the maximum tolerable plasma concentration of lidocaine in human is 0.02 mM (4.7 μg/ml), above this level lidocaine will become toxic. This leads to the conclusion that an inhibitory effect on the P2X7 receptor of at least 50% cannot be reached by administering lidocaine using the traditional intravenous route. This is also true, to a lesser extent, for articaine.

To solve this problem, Applicant formulated lidocaine (with the same rationale applying to articaine) in a high dose/low volume unit dose solution for oromucosal or sublingual administration. The sublingual mucosa is known for its high permeability (20 times more absorption speed compered to human skin) and additionally, the floor of the mouth and oral cavity has a high concentration of lymph nodes, with makes it perfect for targeting the lymphatic system and immune system.

An effective adult unit dose of lidocaine, delivered oromucosally or sublingually, was found to be between 60-100 mg, delivered 4-6 times a day. The objective was to provide an effective dose of lidocaine in the plasma of <4.7 μg/ml.

Given the increased potency of articaine, an equivalent dose would be 40-67 mg, delivered 4-6 times a day. With its' reduced toxicity, equivalent dosing to lidocaine (60-100 mg) 2-4 times a day might also be achieved.

To prove the suitability of such a delivery method a lidocaine formulation was modified from the Astra Zeneca Xylocaine 10% spray formulation by increasing the concentration of lidocaine from 10 to 25%.

Rather than deliver a spray, the formulation was delivered from small, single unit dose, sealed vials (0.4 ml vol) for delivery as drops.

The key was to deliver a unit dose of from 60-100 mg lidocaine in a volume of equal or less than 0.5 ml.

For articaine an equivalent unit dose was 40-67 mg articaine in a volume of equal or less than 0.5 ml. However, due to its' lower toxicity a dose of up to 100 mg could be administered in this volume.

The lidocaine formulation, which is significantly more concentrated than current formulations, comprises greater than 10% by volume of lidocaine, and more preferably greater than 20%, and typically 25%. This has the advantage that it avoids a feeling of fullness in the mouth and accompanied hypersalivation, which is poorly tolerated, and which occurs with standard IV formulations (at a concentration of 10%).

The articaine formulation is also more concentrated than current formulations, comprises greater than 4% by volume of lidocaine, and more preferably greater than 8%, and typically 10% or more. It too can be provided in a unit dose form.

In accordance with a third aspect of the present invention there is provided a method of treating a subject suffering from COVID-19 or an autoimmune disease or a condition in which an immune response caused by the disease or an infection causes a cytokine storm, comprising administering to the subject an effective dose of lidocaine, or a salt thereof, or articaine, or a salt thereof, targeted to the lymphatic system, in a suitable high dose/low volume unit dosage form.

By high dose/low volume unit dosage form is meant the relative concentration of the lidocaine, or a salt thereof, or articaine, or a salt thereof is significantly higher than the existing dosage form (10% for lidocaine or a salt thereof, and 4% for articaine or a salt thereof) with a unit dose volume of 1 ml or less, more preferably 0.5 ml or less.

Preferably the lidocaine, or a salt thereof, or articaine, or a salt thereof, is administered oromucosally or sublingually.

Preferably the unit dose comprises from 60-100 mg of lidocaine or a salt thereof, or 40-100 mg articaine, or a salt thereof.

Preferably the unit dose has a volume of equal or less than 0.5 ml.

The treatment may be, the treatment of COVID-19, Acute respiratory distress syndrome (ARDS). Long-covid or of an autoimmune disease, particularly but not limited to: scleroderma; dermatomyositis; parkinson; and psoriasis.

Most preferably the treatment is for the treatment of long-covid, or the symptoms thereof including: fatigue, moderate to severe breathlessness, pain, insomnia, dyspnoea, dizziness, cramps and or palpitations.

The primary aim of the treatment is to reduce systemic hyperinflammation. This could be determined by measuring biomarkers, such as, for example, interleukin-6, ferritin, leukocytes, neutrophils, lymphocytes, platelets, C-Reactive Protein, procalcitonin, lactate dehydrogenase, aspartate aminotransferase, creatinine, and D-dimer.

Different aspects and embodiments of the invention are further described hereinafter with reference to the detailed description.

### DETAILED DESCRIPTION

The trial formulation is illustrated in Example 1:

### Example 1a

### Formulation

Active
lidocaine: 100 mg

Excipients:
Lipophilic solvent—levomenthol
Polar solvent—ethanol 96% 241 mg/ml,
Co-solvent—polyethylene glycol (Macrogol 400),
Co-solvent—propylene glycol (E 1520)
Sweetener—Saccharin (E 954),
Flavor—banana flavor.
Diluent—water, purified)
Total 0.4 ml

The formulation of the invention was used “off-label” to treat a number of patients to determine its effect as set out in Example 2.

### Example 1b

As example 1a except the active was articaine.

### Example 2

### Patient Trial Data

In the Netherlands, 22 patients were treated with the formulation of Example 1a as follows:

10 patients had COVID-19 symptoms (and were positive and were seen by their general practitioner). All 10 patients presented with early signs of respiratory distress; tachypnea (more than 30 breaths per minute), high fever and dry cough and a decreased Sp02<90. Because they responded well to lidocaine sublingually, they were able to avoid hospitalization for COVID-19);

6 patients had post-COVID respiratory complaints. They were experiencing long-term complaints, ranging from coughing, fatigue and shortness of breath to anxiety and physical limitations.

6 patients suffered with various autoimmune diseases as follows:
1 patient had rheumatoid arthritis.
1 patient had scleroderma;
1 patient had dermatomyositis;
1 patient had morbus Parkinson;
2 patients had psoriasis.

All patients were given the formulation as described in Example 1a above, up to 6 times a day, for up to 2 weeks.

Out of a total of 22 patients, 8 responded to this treatment within hours, and recovered, without problems, within days.

The 10 patients with COVID-19, and 6 with post-COVID respiratory complaints had fully recovered within two weeks.

5 of the 6 patients with the identified autoimmune diseases experienced significant improvement after 2 weeks of treatment and all had no side effects.

Only 1 patient showed minimal improvement, and that was a 56 year old male patient with severe chronic rheumatoid arthritis.

### CONCLUSION

Administration of lidocaine, targeting a subject's lymphatic system, appears effective in treating COVID-19 and long-covid. The sublingual/oromucosal approach is a simple, effective, non-invasive approach. A high concentration low volume unit dose of greater than 10% lidocaine in a small volume, less than 0.5 ml, is optimal.

1. Lidocaine or a salt thereof, or articaine or a salt thereof, for use in treatment of COVID-19 or an autoimmune disease or a condition in which an immune response caused by a disease or infection causes a cytokine storm.

2. The Lidocaine or the salt thereof, or the articaine or the salt thereof, as claimed in claim 1, for use in the treatment of COVID-19 and/or long-covid.

3. The Lidocaine or the salt thereof, or the articaine or the salt thereof, as claimed in claim 1, for use in the treatment of Acute Respiratory Distress Syndrome (ARDS).

4. The Lidocaine or the salt thereof, or the articaine or the salt thereof, as claimed in claim 1, for use in the treatment of the autoimmune disease.

5. The Lidocaine or the salt thereof, or the articaine or the salt thereof, as claimed in claim 4, for use in the treatment of one or more:
scleroderma;
dermatomyositis;
parkinson; and
psoriasis.

6. The Lidocaine or the salt thereof, or the articaine or the salt thereof, as claimed in claim 1, which is formulated for oromucosal or sublingual delivery.

7. The Lidocaine or the salt thereof, or the articaine or the salt thereof, as claimed in claim 6, which is in a high dose/low volume unit form.

8. The Lidocaine or the salt thereof, or the articaine or the salt thereof, as claimed in claim 1, which targets the lymphatic system.

9. The Lidocaine or the salt thereof, or the articaine or the salt thereof, as claimed in claim 1, which comprises, as a unit dose from 40-100 mg of the lidocaine, or the salt thereof, or the articaine, or the salt thereof.

10. A pharmaceutical formulation, comprising: lidocaine, or a salt thereof, or articaine, or a salt thereof, which is formulated to facilitate uptake by a lymphatic system with one or more suitable excipients.

11. The pharmaceutical formulation as claimed in claim 10, wherein the one or more suitable excipients comprise a lipophilic excipient.

12. The pharmaceutical formulation as claimed in claim 11, wherein the lipophilic excipient is menthol.

13. The pharmaceutical formulation as claimed in claim 10, further comprising a polar solvent.

14. The pharmaceutical formulation as claimed in claim 13, wherein the polar solvent is ethanol.

15. The pharmaceutical formulation as claimed in claim 10, further comprising one or more co-solvents.

16. The pharmaceutical formulation as claimed in claim 15, wherein the one or more co-solvents comprise polyethylene and polypropylene glycol.

17. The pharmaceutical formulation as claimed in claim 10, further comprising a sweetener and a flavourant.

18. The pharmaceutical formulation as claimed in claim 10, comprising from 60-100 mg of the lidocaine, or the salt thereof, or from 40-100 mg of the articaine, or the salt thereof, in a unit dose.

19. The pharmaceutical formulation as claimed in claim 10, comprising a unit dose with a volume of less than or equal to 0.5 ml.

20. The pharmaceutical formulation as claimed in claim 10, comprising at least a 20% by weight solution of the lidocaine, or the salt thereof, in a volume of less than or equal to 0.5 ml.

21. The pharmaceutical formulation as claimed in claim 10, comprising at least a 10% by weight solution of the articaine, or the salt thereof, in a volume of less than or equal to 0.5 ml.

22. A method of treating a subject suffering from COVID-19 or an autoimmune disease or a condition in which an immune response caused by the disease or an infection causes a cytokine storm, comprising: administering to the subject an effective dose of lidocaine, or a salt thereof, or articaine, or a salt thereof, targeted to a lymphatic system, in a suitable high dose/low volume unit dosage form.

23. The method as claimed in claim 22, wherein the lidocaine, or the salt thereof, or the articaine, or the salt thereof, is administered oromucosally or sublingually.

24. The method as claimed in claim 22, wherein the lidocaine, or the salt thereof, or the articaine, or the salt thereof, is provided in a unit dose in an amount of from 40-100 mg.

25. The method as claimed in claim 24, wherein the unit dose has a volume of less than or equal to 0.5 ml.

26. The method as claimed in claim 22, which is for the treatment of COVID-19 or long-covid.

27. The method as claimed in claim 22, which is for the treatment of Acute respiratory distress syndrome (ARDS).

28. The method as claimed in claim 22, which is for the treatment of an autoimmune disease.

29. The method as claimed in claim 28, wherein the autoimmune disease is selected from:
scleroderma;
dermatomyositis;
parkinson; and
psoriasis.
